(19) United States
(12) Patent Application Publication
(10) Pub. No.: US 2005/0203488 A1
(43) Pub. Date: Sep. 15, 2005

(54) Apparatus and methods for mapping out endoluminal gastrointestinal surgery

(75) Inventors: Kenneth J. Michlitsch, Livermore, CA (US); Vahid Saadat, Saratoga, CA (US); Rodney Brenneman, San Juan Capistrano, CA (US); Tracy Maahs, Rancho Santa Margarita, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI MEDICAL INC., San Clemente, CA (US)

(21) Appl. No.: 10/797,910

(22) Filed: Mar. 9, 2004

(51) Int. Cl. A61B 17/00; A61B 19/00

(52) U.S. Cl. 606/1; 128/898

(57) ABSTRACT

Apparatus and methods are provided for mapping out endoluminal gastrointestinal surgery, including endoluminal gastric reduction. Mapping is achieved by locally marking the interior of the gastrointestinal lumen at specified locations. In a first embodiment, the surgical map comprises localized RF scarring or mucosal ablation. In an alternative embodiment, the map comprises pegs. In another alternative embodiment, the map comprises dye and/or spheres injected into at least the submucosa. As a still further alternative, the map may comprise the shaft of an endoluminal surgical tool having specified dimensions and/or color-coding, etc. In another alternative embodiment, the map may be formed from surgical mesh. In one preferred embodiment, placement of the map is accurately achieved by approximating the interior of the stomach with an endoluminal support via suction ports and/or via an inflatable member disposed along the support. Methods of using apparatus of the present invention are provided.

### CROSS-REFERENCES TO RELATED APPLICATIONS

This application contains subject matter related to, but does not claim continuing status from, the following prior applications: U.S. patent application Ser. No. 10/735,030, filed Dec. 12, 2003, which is a Continuation-In-Part of U.S. patent application Ser. No. 10/672,375, filed Sep. 23, 2003, which claims the benefit of the filing date of U.S. provisional patent application Ser. No. 60/500,627, filed Sep. 5, 2003; U.S. patent application Ser. No. 10/612,170, filed Jul. 1, 2003, and Ser. No. 10/639,162, filed Aug. 11, 2003; both of which claim the benefit of the filing date of U.S. provisional patent application Ser. No. 60/433,065, filed Dec. 11, 2002; U.S. patent application Ser. No. 10/173,203, filed Jun. 13, 2002; U.S. patent application Ser. No. 10/458,060, filed Jun. 9, 2003, which is a Continuation-In-Part of U.S. patent application Ser. No. 10/346,709, filed Jan. 15, 2003, and which claims the benefit of the filing date of U.S. provisional patent application Ser. No. 60/471,893, filed May 19, 2003; and U.S. patent application Ser. No. 10/288,619, filed Nov. 4, 2002, which is a Continuation-In-Part of U.S. patent application Ser. No. 09/746,579, filed Dec. 20, 2000, and a Continuation-In-Part of U.S. patent application Ser. No. 10/188,509, filed Jul. 3, 2002, which is a Continuation-In-Part of U.S. patent application Ser. No. 09/898,726, filed Jul. 3, 2001, which is a Continuation-In-Part of U.S. patent application Ser. No. 09/602,436, filed Jun. 23, 2000, which claims the benefit of the filing date of U.S. provisional patent application Ser. No. 60/141,077, filed Jun. 25, 1999. All of these applications are incorporated herein by reference in their entireties.

### BACKGROUND OF THE INVENTION

Field of the Invention. The present invention relates to methods and apparatus for mapping out endoluminal gastrointestinal (“GI”) surgery. More particularly, the present invention relates to methods and apparatus for mapping out endoluminal gastric reduction.

Morbid obesity is a serious medical condition pervasive in the United States and other countries. Its complications include hypertension, diabetes, coronary artery disease, stroke, congestive heart failure, multiple orthopedic problems and pulmonary insufficiency with markedly decreased life expectancy.

Several open and laparoscopic surgical techniques have been developed to treat morbid obesity, e.g., bypassing an absorptive surface of the small intestine, or reducing the stomach size. These procedures are difficult to perform in morbidly obese patients because it is often difficult to gain access to the digestive organs. In particular, the layers of fat encountered in morbidly obese patients make difficult direct exposure of the digestive organs with a wound retractor, and standard laparoscopic trocars may be of inadequate length. In addition, previously known open surgical procedures may present numerous life-threatening post-operative complications, and may cause atypical diarrhea, electrolytic imbalance, unpredictable weight loss and reflux of nutritious chyme proximal to the site of the anastomosis.

Applicant has previously described methods and apparatus for endoluminally reducing a patient's stomach, for example, in co-pending U.S. patent application Ser. No. 10/735,030, filed Dec. 12, 2003, which is incorporated herein by reference in its entirety. That application describes an endoluminal technique for creating a small pouch below the gastroesophageal junction to limit food intake and promote a feeling of satiety. The endoluminal pouch acts in a manner similar to a Vertical Banded Gastroplasty (“VBG”).

The gastrointestinal lumen includes four tissue layers, wherein the mucosa layer is the top (innermost) tissue layer, followed by connective tissue, the muscularis layer and the serosa layer. One problem with endoluminal gastrointestinal reduction systems is that the anchors (or staples) must engage at least the muscularis tissue layer in order to provide a proper foundation, since the mucosa and connective tissue layers tend to stretch elastically under the tensile loads imposed by normal movement of the stomach wall during ingestion and processing of food. Applicant's techniques for endoluminal VBG reduction address this concern by reconfiguring the stomach lumen via engagement of at least the muscularis layer of tissue.

It is expected that proper placement of anchors or suture to achieve such endoluminal VBG will present significant challenges to a medical practitioner, due, for example, to the limited working space, as well as the limited visualization provided by, e.g., an endoscope or fiberscope. U.S. Pat. No. 6,558,400 to Deem et al. describes methods and apparatus for marking the interior of the stomach from the esophagus to the pylorus to map out an endoluminal reduction procedure. Marking is achieved with dye channeled through ports in a marking device or bougie. The bougie optionally may comprise suction ports for evacuating the stomach about the bougie, at which point the dye may be injected to stain the stomach along points that contact the dye ports. The stomach then may be insufflated for performing the endoscopic reduction procedure utilizing the map provided by the dye marks stained onto the stomach mucosa.

A significant drawback of the marking technique described by Deem et al. is that dyes have a tendency to spread and are very difficult to localize, especially in a fluid environment such as that which contacts the mucosa layer of the stomach. As such, it is expected that dye that does not penetrate beyond the mucosa will provide an inaccurate and/or unstable map for performing endoscopic gastric reduction. This, in turn, may yield an incorrectly sized or poorly sealed stomach pouch, which may render the procedure ineffective in facilitating weight loss and/or may result in dangerous complications.

In view of the aforementioned limitations, it would be desirable to provide methods and apparatus for mapping out endoluminal gastrointestinal surgery that may be readily localized.

It would be desirable to provide methods and apparatus for mapping out endoluminal gastrointestinal surgery that enhance accuracy.

It also would be desirable to provide methods and apparatus that enhance stability of the surgical map.

### BRIEF SUMMARY OF THE INVENTION

In view of the foregoing, it is an object of the present invention to provide methods and apparatus for mapping out endoluminal gastrointestinal surgery that may be readily localized.

It is another object of the present invention to provide methods and apparatus for mapping out endoluminal gastrointestinal surgery that enhance accuracy.

It is an additional object of this invention to provide methods and apparatus for mapping out endoluminal gastrointestinal surgery that enhance stability of the surgical map.

These and other objects of the present invention are accomplished by providing apparatus and methods for marking the interior of the gastrointestinal lumen. In a first embodiment, the surgical map comprises localized RF scarring or mucosal ablation. In an alternative embodiment, the map comprises pegs, e.g. colored pegs, which may be biodegradable, e.g. fabricated from polyglycolic acid. Alternatively, the pegs may comprise one or more corkscrews advanced into tissue surrounding the GI lumen. In yet another alternative embodiment, the map comprises dye injected into at least the submucosa. The dye may be fluorescent or of varying colors. Alternatively, the dye may be disposed within nanospheres or microspheres implanted submucosally. In addition, or as an alternative, to dye spheres, the spheres may be magnetic, heat-able ferromagnetic or Curie point, plastic and inert, radiopaque, etc. As a still further alternative, the map may comprise the shaft of an endoluminal surgical tool having specified dimensions and/or color-coding, etc. In another alternative embodiment, the map may be formed from surgical mesh. Additional mapping apparatus will be apparent.

In one preferred embodiment, placement of the map is accurately achieved using suction ports and/or an inflatable member disposed along an endoluminal support, such as a shaft or other tool associated with the endoluminal GI surgery. When using suction, the stomach may be deflated about the support prior to deployment of the surgical map. When using an inflatable member, the inflatable member may be inflated to contact tissue prior to deployment of the map. As will be apparent, a combination of suction and inflation may be used to properly orient tissue prior to mapping.

Methods of using the apparatus of the present invention also are provided.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is an isometric view of a first embodiment of apparatus of the present invention configured to map out an endoluminal gastrointestinal surgery, the apparatus comprising suction ports and RF elements configured to selectively scar or ablate the interior wall of the GI lumen;

FIGS. 2A-2C are, respectively, a side view, partially in section; a cross-sectional detail view along view line A-A in FIG. 2A; and a side-sectional view along view line B-B of FIG. 2A; illustrating a method of using the apparatus of FIG. 1 to map out an endoscopic stomach reduction procedure;

FIG. 3 is a schematic view of an alternative embodiment of the apparatus of FIG. 1 that is configured to engage tissue via an inflatable member.

FIGS. 4A-4C are schematic views of alternative apparatus for mapping out an endoluminal GI surgery with pegs;

FIG. 5 is a schematic view of additional alternative apparatus for mapping out an endoluminal GI surgery, the apparatus comprising a catheter configured to locally deliver a marking element at least submucosally;

FIGS. 6A and 6B are, respectively, a side view and a side detail view, both partially in section, illustrating a method of using the apparatus of FIG. 5 to map out an endoluminal GI surgery;

FIGS. 7A-7D are, respectively, a side view, partially in section; side-sectional detail views along section line C-C in FIG. 7A; and a side-sectional view; illustrating a method of mapping out an endoluminal GI surgery with the shaft of an endoluminal surgical tool having specified characteristics;

FIGS. 8A-8C are, respectively, a side view, partially in section; a cross-sectional detail view along section line D-D in FIG. 8A; and a side-sectional view along section line E-E in FIG. 8A, illustrating a method of mapping out endoluminal GI surgery with surgical mesh; and

FIG. 9 is a side view, partially in section, illustrating a method of mapping out endoluminal GI surgery with an RF marking element disposed on an inflatable member.

### DETAILED DESCRIPTION OF THE INVENTION

The present invention relates to methods and apparatus for mapping out endoluminal gastrointestinal (“GI”) surgery. More particularly, the present invention relates to methods and apparatus for mapping out endoluminal gastric reduction.

Applicant has previously described methods and apparatus for endoluminally forming and securing GI tissue folds, for example, in U.S. patent application Ser. No. 10/735,030, filed Dec. 12, 2003, which is incorporated herein by reference. Such methods and apparatus may be used to reduce or partition the effective cross-sectional area of a GI lumen, e.g., to treat obesity by approximating the walls of the stomach to narrow the stomach lumen and/or create a pouch or endoluminal Vertical Banded Gastroplasty (“VBG”), thus promoting a feeling of satiety and reducing the area for food absorption. However, as discussed previously, it is expected that proper placement of anchors or suture to form and secure such endoluminal VBG will present significant challenges to a medical practitioner, due, for example, to the limited working space, as well as the limited visualization provided by, e.g., an endoscope or fiberscope.

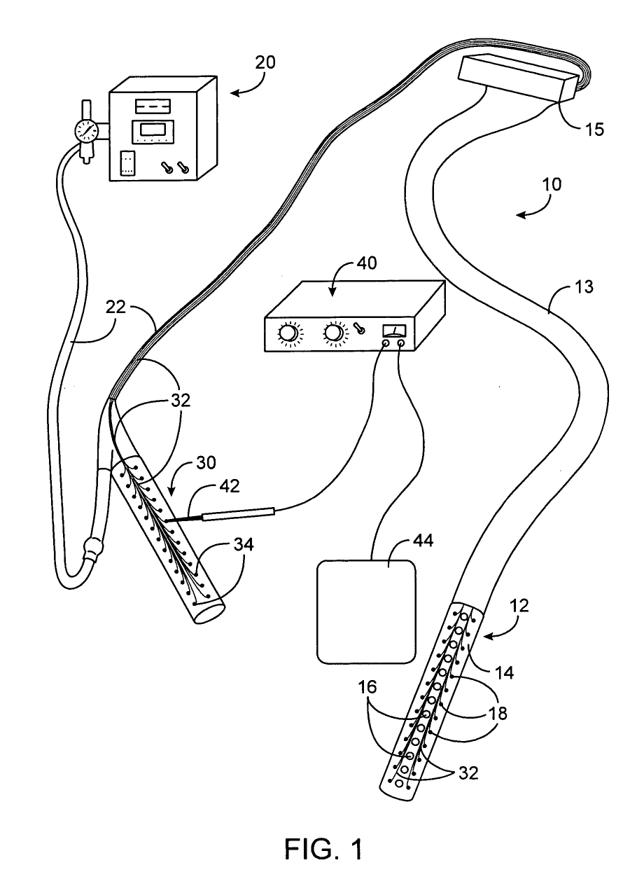

Referring now to FIG. 1, a first embodiment of apparatus for mapping out endoluminal GI surgery in accordance with the present invention is described. Apparatus 10 comprises endoluminal support 12 having shaft 14 with one or more, e.g. a plurality, of suction ports 16 and one or more, e.g. a plurality, of radiofrequency (“RF”) marking electrodes 18 disposed along the length of the shaft. Suction ports 16 are proximally coupled to suction pump 20 via tubing 22. Likewise, each RF marking electrode 18 is connected to switching station 30 via a wire 32. As seen in FIG. 1, wires 32 optionally may be routed through tubing 22 over at least a portion of their length. Switching station 30 comprises electrical contacts 34 that are electrically connected to RF marking electrodes 18 via wires 32. Apparatus 10 further comprises RF generator 40, which is configured to actuate electrodes 18 via switching station 30. RF generator 40 comprises positive electrode 42 and negative or ground electrode 44. RF generator 40 may comprise a commercially available RF generator, per se known, for example, such as those distributed by Everest Medical of Maple Grove, Minn.

In use, endoluminal support 12 may be endoluminally advanced within a GI lumen, e.g. a patient's stomach. Actuation of suction pump 20 from outside the patient draws suction through tubing 22 and suction ports 16, thereby bringing luminal GI tissue into contact with shaft 14 of endoluminal support 12. Meanwhile, negative electrode 44 of RF generator 40 may be placed exterior to the patient, e.g., on the patient's chest, or on a metal operating table just under the patient's back while the patient lies on the table. As will be apparent, negative electrode 44 alternatively may be coupled to endoluminal support 12, for example, along shaft 14 at a location radially distant from RF electrodes 18. Positive electrode 42 may be selectively connected to any of the plurality of electrical contacts 34 of switching station 30, as desired, to actuate specified RF marking electrodes 18.

Actuation of electrodes 18 via RF generator 40 acts to locally burn, singe, cut, ablate, scar or otherwise injure tissue in contact with the electrodes along shaft 14 of endoluminal support 12, thereby leaving identifiable marks on the surface of the tissue that may be used to map out an endoluminal GI surgery. As will be apparent to those of skill in the art, the pattern of electrodes 18 and suction ports 16 about shaft 14 of endoluminal support 12 may be altered as desired to facilitate formation of surgical maps having varying characteristics. Likewise, the shape or orientation of shaft 14 may be altered.

Switching station 30 facilitates actuation of individual electrodes 18, as well as actuation of any combination of the individual electrodes, including simultaneous actuation of all the electrodes. Such selective actuation is dependent upon which electrical contact(s) 34 are connected to positive electrode 42 of RF generator 40 when the generator is energized. As will be apparent, switching station 30 optionally may be omitted, and wires 32 may couple RF electrodes 18 directly to RF generator 40.

Endoluminal support 12 optionally may comprise one or more working lumens (not shown) for advancing additional surgical instruments through the endoluminal support. Additionally or alternatively, endoluminal support 12 optionally may comprise proximal shaft 13 that is steerable and/or rigidizable or shape-lockable, e.g. via pull wires actuated through handle 15. Rigidizable shafts are described, for example, in Applicant's co-pending U.S. patent application Ser. No. 10/735,030, filed Dec. 12, 2003, which is incorporated herein by reference: When utilizing a steerable, rigidizable shaft, endoluminal support 12 may be steered into proper position within a GI lumen, rigidized to maintain its position, and then actuated as described above to mark tissue and map out endoluminal GI surgery.

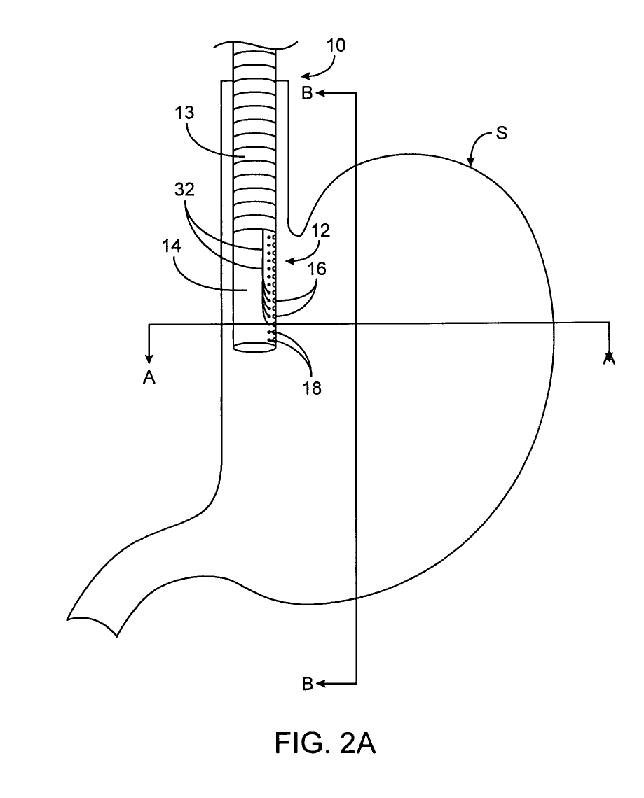

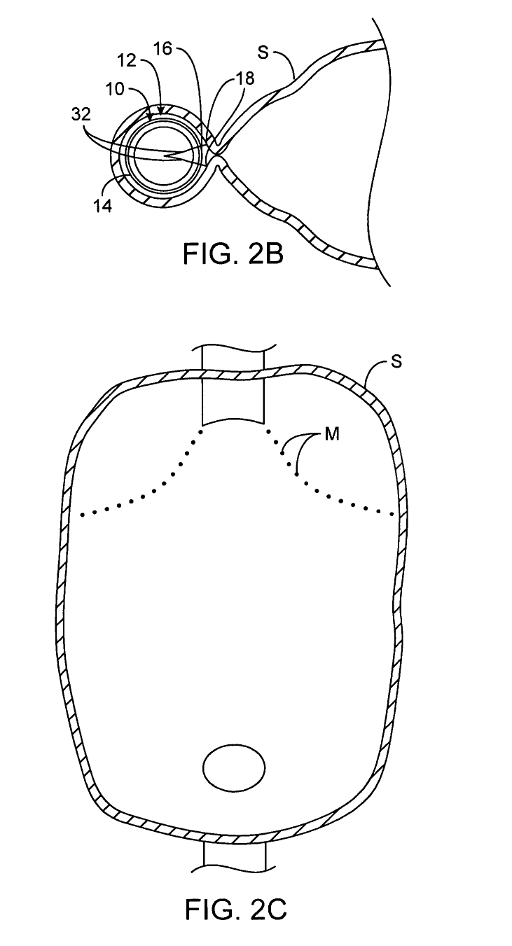

With reference now to FIG. 2, in conjunction with FIG. 1, a method of using the apparatus of FIG. 1 to map out an endoscopic stomach reduction procedure is described. In FIG. 2A, endoluminal support 12 of apparatus 10 is endoluminally advanced down a patient's throat into the patient's stomach S. Suction ports 16 and RF electrodes 18 are oriented towards the greater curvature of stomach S. Negative electrode 44 of RF generator 40 is placed exterior to the patient in close proximity to shaft 14 of apparatus 10 (not shown). Suction pump 20 is then actuated to pull suction through suction ports 16 and deflate the stomach about shaft 14 of endoluminal support 12, as in FIG. 2B. Positive electrode 42 of RF generator 40 is connected to one or more electrical contacts 34 of switching station 30, and the RF generator is actuated to locally mark the interior wall of stomach S with marks M at locations in contact with actuated electrodes 18.

Once RF electrodes 18 have been actuated in a desired pattern and for a desired duration at a desired intensity, RF generator 40 is turned off and/or positive electrode 42 is disconnected from switching station 30. As seen in FIG. 2C, stomach S then may be insufflated, e.g., via air injected through suction ports 16. Marks M burned or ablated into the mucosa of the stomach may be used as a map for performing endoluminal stomach reduction, for example, as described in Applicant's co-pending U.S. patent application Ser. No. 10/735,030.

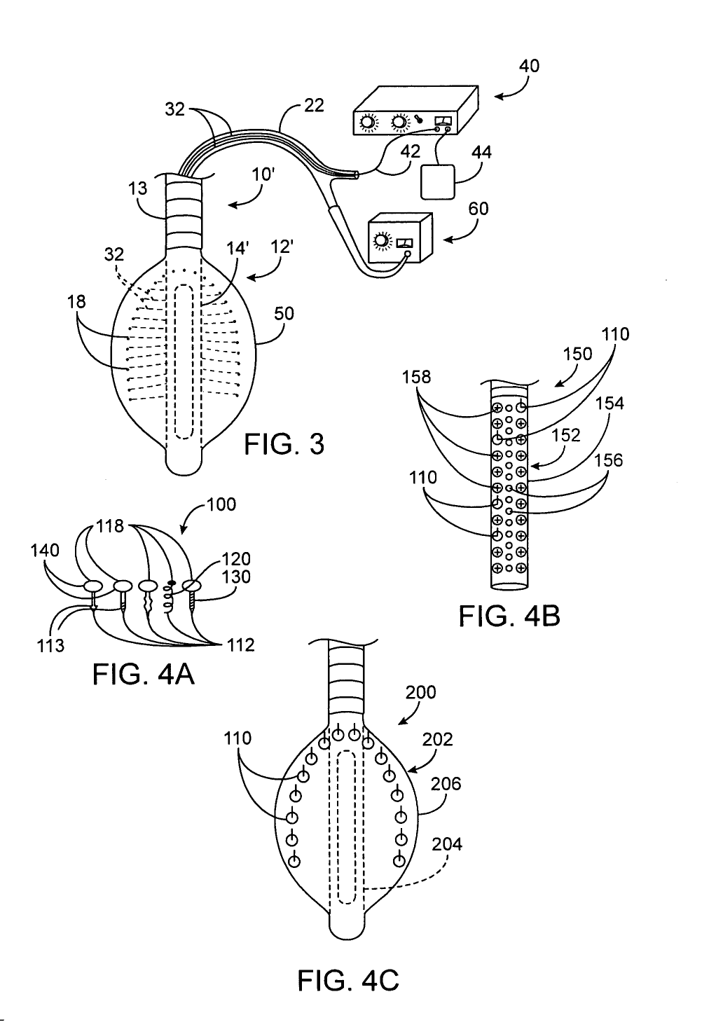

Referring now to FIG. 3, an alternative embodiment of apparatus 10 is described wherein the suction elements have been replaced with inflatable elements. Endoluminal support 12′ of apparatus 10′ comprises inflatable member 50 coupled to shaft 14′. Inflatable member 50 is illustratively shown at least partially inflated in FIG. 3. RF electrodes 18 are coupled to the exterior of the inflatable member in an appropriate pattern, and tubing 22 couples inflatable member 50 to inflation source 60, e.g., a compressor or a syringe. In FIG. 3, switching station 30 has been eliminated, and RF electrodes 18 have been connected directly to positive electrode 42 of RF generator 40 via wire(s) 32. In this manner, actuation of RF generator 40 energizes all electrodes 18 simultaneously.

In use, endoluminal support 12′ is endoluminally advanced within a patient's stomach and/or GI lumen. Inflatable member 50 is inflated via inflation medium transferred from source 60 through tubing 22 to the inflatable member. The inflatable member conforms to the interior profile of the GI lumen, thereby bringing RF electrodes 18 into contact with the interior wall of the lumen. The electrodes then may be actuated as described previously to form marks M for mapping out an endoluminal GI surgery. As will be apparent, a combination of suction and inflation may be used to properly orient tissue prior to marking and mapping.

Referring now to FIG. 4, alternative apparatus for mapping out an endoluminal GI surgery is described. As seen in FIG. 4A, apparatus 100 comprises a plurality of pegs 110 that are configured to engage tissue and act as a map for endoluminal GI surgery. The pegs optionally may comprise sharpened distal ends 112 configured to penetrate tissue. Pegs 110 may also comprise optional barbs, hooks, etc. 113 to maintain the pegs in the tissue after penetration. The pegs may be endoluminally implanted at appropriate locations, then visualized to provide a map for the GI surgery. They preferably are colored to enhance visibility, and optionally may be provided in a variety of colors, shapes, sizes, etc. to provide additional mapping information. Pegs 110 preferably are biodegradable, e.g., fabricated from polyglycolic acid. Pegs 110 optionally may comprise a plurality of corkscrews 120. Corkscrews may require less force to advance into tissue, as compared to pegs with substantially straight shafts having sharpened distal ends 112. The rotational motion used to advance corkscrews applies enhanced force within the plane of tissue, as opposed to perpendicular to the plane. As an alternative to corkscrews, screws 130 may be provided. Alternatively tacks 140 may be provided. Additional pegs will be apparent.

FIGS. 4B and 4C illustrate modified embodiments of previously described apparatus 10 and 10′, respectively, that are configured to deliver and deploy pegs 110 of apparatus 100. In FIG. 4B, apparatus 150 comprises endoluminal support 152 having suction ports 156 disposed along shaft 154. Suction ports 156 are coupled to suction pump 20 via tubing 22, as described previously. Pegs 110 are disposed in channels 158 along shaft 154 and may be deployed from the channels into tissue when tissue is disposed about the shaft, e.g., via suction drawn through ports 156. Advancement of the pegs into tissue may be achieved via pushrods, e.g. torque-able pushrods (not shown). In FIG. 4B, a few pegs illustratively are shown advanced out of channels 158.

In FIG. 4C, apparatus 200 comprises endoluminal support 202 having inflatable member 206 disposed along shaft 204. Pegs 110 are lightly adhered to the surface of inflatable member 206, such that the pegs may engage tissue and decouple from the inflatable member upon inflation of the inflatable member into contact with the tissue. Various mechanisms may be provided for releasably securing pegs 110 to the surface of inflatable member 206, for example, adhesives, electromagnets, fuse mechanisms, etc.

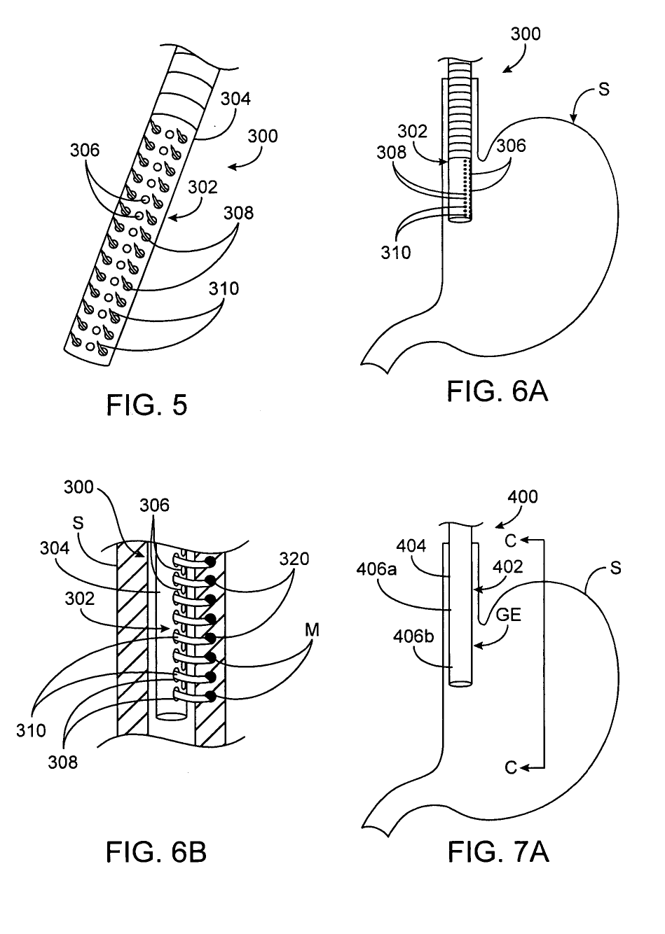

With reference now to FIG. 5, alternative apparatus for mapping out an endoluminal GI surgery is described, the apparatus comprising a marking element in combination with a catheter configured to locally deliver the marking element at least submucosally. Apparatus 300 comprises endoluminal support 302 having suction ports 306 disposed along shaft 304. Suction ports 306 are coupled to suction pump 20 via tubing 22, as described previously. Apparatus 300 further comprises injection channels 308 having retractable needles 310. Needles 310 are illustratively shown at least partially extended in FIG. 5.

In use, endoluminal support 302 may be advanced within a GI lumen with needles 310 retracted. Suction then may be drawn through ports 306 to bring tissue into proximity with channels 308. Needles 310 then may be extended into the tissue to penetrate the tissue. When conducting endoluminal gastric procedures, the needles are configured to penetrate the tissue at least submucosally. Upon penetration of tissue by needles 310, marking elements may be injected into the tissue below the surface through the needles.

Illustrative subsurface or submucosal marking elements include, but are not limited to, dyes, fluorescent dyes and colored dyes. As described in U.S. Pat. No. 6,558,400 to Deem et al., which is incorporated herein by reference, marking dyes may comprise, for example, methylene blue, thionine, acridine orange, acridine yellow, acriflavine, quinacrine and its derivatives, brilliant green, gentian violet, crystal violet, triphenyl methane, bis naphthalene, trypan blue, and trypan red. U.S. Pat. No. 6,558,400 describes using such dyes to mark or stain the interior lining of the stomach. However, that reference does not describe injecting such dyes submucosally. Submucosal injection is expected to enhance localization, stability and accuracy, as compared to mucosal staining.

Additional subsurface/submucosal marking elements include, for example, saline or bulking agents, e.g. collagen, to achieve geometric marking/mapping via localized protrusion of the mucosa. As yet another alternative, nanospheres or microspheres may be utilized, e.g. colored spheres or dye-filled spheres. In addition, or as an alternative, to dye spheres, the spheres may be magnetic, heat-able ferromagnetic or Curie point, plastic and inert, bioresorbable, radiopaque, etc. Curie point materials may be heated to a known temperature via an external electromagnetic field, for example, to cause local ablation, inflammation or scar formation, etc. Such local marking may be used to map out an endoluminal GI surgery.

With reference now to FIG. 6, a method of using the apparatus of FIG. 5 to map out an endoluminal stomach reduction is described. In FIG. 6A, endoluminal support 302 of apparatus 300 is endoluminally advanced down a patient's throat into the patient's stomach S. Suction ports 306 and injection channels 308, having needles 310 retracted therein, are oriented towards the greater curvature of stomach S. Suction pump 20 is actuated to pull suction through suction ports 306 and deflate the stomach about shaft 304 of endoluminal support 302. Needles 310 are advanced out of injection channels 308 to penetrate tissue in proximity to the channels, as seen in FIG. 6B. The distal tips of needles 310 are disposed submucosally. Marking elements 320, which may comprise dye, spheres, etc., are injected submucosally through needles 310, thereby locally and submucosally marking the interior wall of stomach S with marks M at locations penetrated by the needles. Needles 310 are removed from the wall of stomach S, and suction pump 20 is deactivated, leaving a map of marks M within the wall of the stomach for endoluminal gastric reduction.

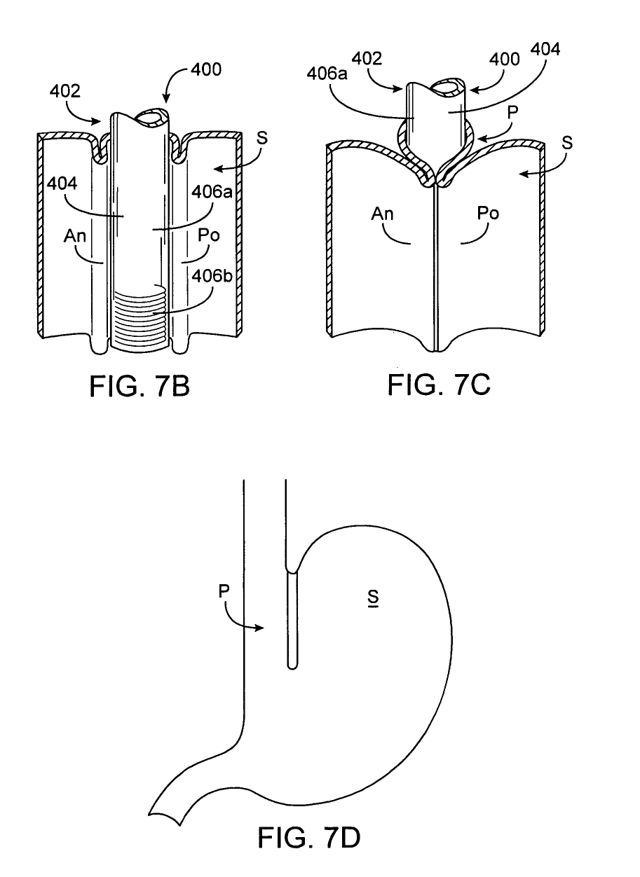

Referring now to FIG. 7, a method of mapping out an endoluminal gastric reduction with the shaft of an endoluminal surgical tool having specified dimensions and/or color-coding is described. Apparatus 400 comprises surgical tool 402 having shaft 404 of specified dimensions appropriate for forming an endoluminal VBG, for example, a diameter of about 1 cm. Shaft 404 optionally may also comprise a plurality of variously colored or patterned sections to provide additional mapping instructions or guideposts for a medical practitioner. In FIG. 7, shaft 404 illustratively comprises first and second sections 406a and 406b having different surface patterns.

In FIG. 7A, shaft 404 is disposed in stomach S inferior to the patient's gastroesophageal junction GE. In FIG. 7B, anterior An and posterior Po tissue ridges are formed on either side of shaft 404, for example, utilizing apparatus and methods described in Applicant's co-pending U.S. patent application Ser. No. 10/735,030, which is incorporated herein by reference. The ridges are then wrapped around shaft 404 and secured to one another, as in FIG. 7C. In FIG. 7D, removal of shaft 404 leaves pouch P in stomach S, thereby completing endoluminal VBG. Apparatus 400 maps out the endoluminal VBG procedure by providing the medical practitioner with visual cues as to proper location for formation of the anterior and posterior ridges, as well as proper sizing for pouch P upon approximation of the ridges.

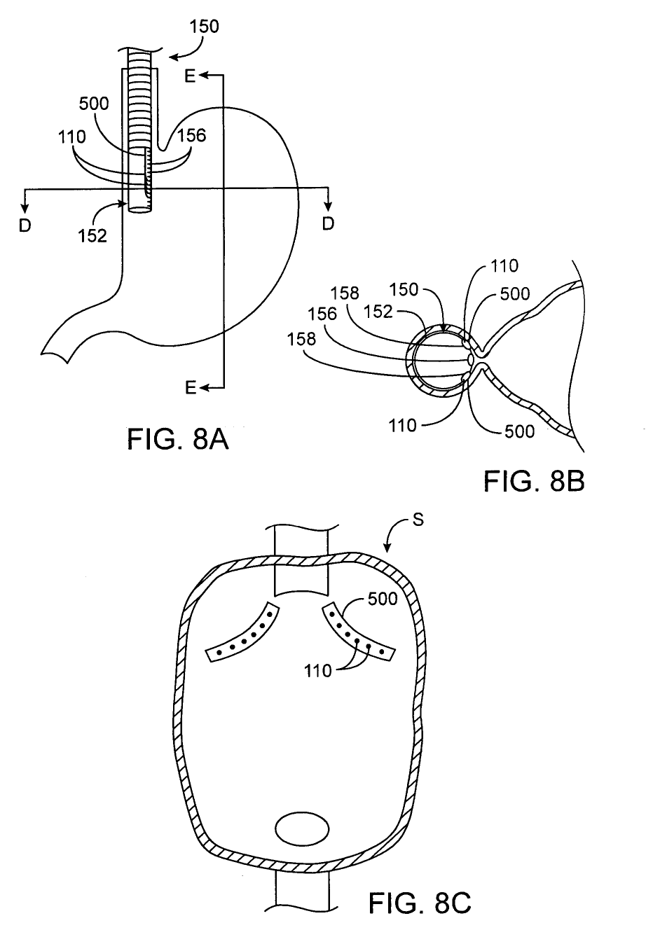

With reference to FIG. 8, a method of using surgical mesh to map out endoluminal GI surgery is described. In FIG. 8, apparatus 150 and pegs 110 of FIG. 4 are used in conjunction with surgical mesh strips 500, which are coupled to pegs 110 disposed in channels 158. As seen in FIG. 8A, endoluminal support 152 of apparatus 150 is advanced into a patient's stomach S. Suction is then drawn through ports 156 via pump 20, such that the stomach deflates about shaft 154 of device 152, as seen in FIG. 8B. Pegs 110 are advanced out of channels 158 into the wall of the stomach, thereby tacking surgical mesh strips 500 to the wall. As seen in FIG. 8C, suction is deactivated and apparatus 150 is removed from the patient, leaving strips 500 as a surgical map disposed on the anterior and posterior of stomach S. The strips may be used to map out the formation of ridges and a pouch in a manner similar to that described with respect to FIG. 7.

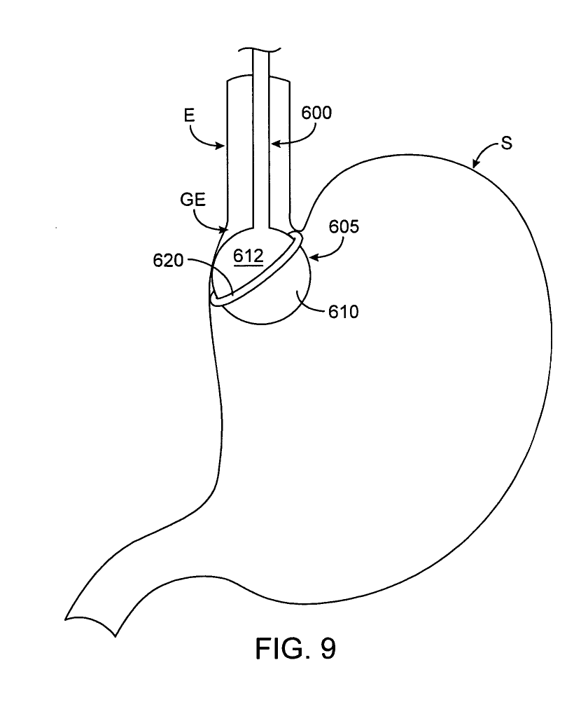

With reference to FIG. 9, a method of mapping out endoluminal gastric reduction or restriction with an RF marking electrode disposed on an inflatable member is described. In FIG. 9, apparatus 600 comprises endoluminal support 605 having inflatable member 610 with positive RF marking electrode 620 disposed in a ring about the surface of the balloon. Ring electrode 320 preferably is flexible and ‘painted’ on the exterior of inflatable member 610, for example, with a conductive paint, such as a silver paint. In this manner, electrode 620 may accommodate changes in dimension as inflatable member 610 is inflated or deflated.

Inflatable member 610 is coupled to an inflation source, such as previously described inflation source 60 of FIG. 3, for inflating and deflating the member. Furthermore, RF marking electrode 620 is electrically connected to an RF generator, such as RF generator 40 of FIG. 3, which further is coupled to a negative electrode, e.g. electrode 44 of FIG. 3, that preferably is disposed external to the patient. Suction elements also may be provided, for example, suction ports 16 in communication with suction pump 20, as in FIG. 1.

In FIG. 9, endoluminal support 605 of apparatus 600 has been advanced endoluminally through esophagus E into stomach S. Inflatable member 610 then has been inflated, e.g. via inflation source 60, with a known fluid volume. Endoluminal support 605 has been retracted proximally until inflatable member 610 abuts gastroesophageal junction GE.

Ring electrode 620 then is activated, e.g. via RF generator 40, to locally singe, burn or otherwise mark the interior of stomach S. After marking, electrode 620 is deactivated, inflatable member 610 is deflated, and endoluminal support 605 of apparatus 600 is removed from stomach S, thereby leaving a map within the stomach for conducting endoluminal gastric reduction or restriction. Advantageously, the volume of fluid disposed in upper left portion 612 of inflatable member 610 (the portion of the inflatable member disposed proximal of marking electrode 620) during activation of electrode 620 substantially defines the mapped out volume of a pouch that may be formed utilizing the map provided by apparatus 600. In this manner, a stomach pouch of specified volume may be accurately formed. As will be apparent, prior to marking stomach S via activation of electrode 620, the stomach optionally may be deflated, e.g. via suction, in order to better approximate stomach tissue against inflatable member 610 and electrode 620.

Although preferred illustrative embodiments of the present invention are described hereinabove, it will be apparent to those skilled in the art that various changes and modifications may be made thereto without departing from the invention. For example, when utilizing an RF endoluminal support in accordance with the present invention, the negative electrode(s) may be placed internally while the positive electrode(s) are disposed external to the patient. It is intended in the appended claims to cover all such changes and modifications that fall within the true spirit and scope of the invention.

1. Apparatus for mapping out endoluminal gastrointestinal surgery, the apparatus comprising:
an endoluminal support configured for endoluminal placement within a gastrointestinal lumen; and
a marking device disposed on the support, the marking device configured to submucosally mark the gastrointestinal lumen.

2. The apparatus of claim 1, further comprising an approximation element configured to approximate the support and a mucosal surface of the gastrointestinal lumen.

3. The apparatus of claim 2, wherein the approximation element is disposed on the endoluminal support.

4. The apparatus of claim 2, wherein the approximation element comprises an element chosen from the group consisting of suction ports, inflation elements and combinations thereof.

5. The apparatus of claim 1, wherein the submucosal marking device comprises needles configured to penetrate mucosal tissue.

6. The apparatus of claim 5, wherein the needles are configured to inject marking elements submucosally.

7. The apparatus of claim 5, wherein the marking elements are chosen from the group consisting of dyes, fluorescent dyes, colored dyes, saline, bulking agents, collagen, spheres, nanospheres, magnetic materials, ferromagnetic materials, Curie point materials, plastic materials, inert materials, radiopaque materials, bioresorbable materials and combinations thereof.

8. Apparatus for mapping out endoluminal gastrointestinal surgery, the apparatus comprising:
an endoluminal support configured for endoluminal placement within a gastrointestinal lumen; and
a radiofrequency element on the support for marking the gastrointestinal lumen.

9. The apparatus of claim 8 further comprising an approximation element configured to approximate an interior of the gastrointestinal lumen and the endoluminal support.

10. The apparatus of claim 9, wherein the approximation element is disposed on the endoluminal support.

11. The apparatus of claim 8, wherein the radiofrequency element comprises at least one electrode, the at least one electrode disposed on a surface of the endoluminal support and coupleable to a radiofrequency generator.

12. The apparatus of claim 9, wherein the approximation element comprises an element chosen from the group consisting of suction ports, inflation elements and combinations thereof.

13. Apparatus for mapping out endoluminal gastrointestinal surgery, the apparatus comprising:
an endoluminal support configured for endoluminal placement within a gastrointestinal lumen; and
a marking device disposed on the support for marking the gastrointestinal lumen with pegs.

14. The apparatus of claim 13 further comprising an approximation element configured to approximate an interior of the gastrointestinal lumen and the endoluminal support.

15. The apparatus of claim 14, wherein the approximating element is disposed on the endoluminal support.

16. The apparatus of claim 13, wherein the marking device further comprises surgical mesh.

17. Apparatus for mapping out endoluminal gastrointestinal surgery, the apparatus comprising:
an endoluminal support configured for endoluminal placement within a gastrointestinal lumen; and
indicia on the endoluminal support which are visible to provide a map of the endoluminal gastric reduction when the endoluminal support is present in the gastrointestinal lumen.

18. The apparatus of claim 17 further comprising an approximation element configured to approximate an interior of the gastrointestinal lumen and the endoluminal support.

19. The apparatus of claim 15, wherein the approximating element is disposed on the endoluminal support.

20. The apparatus of claim 17, wherein the indicia are chosen from the group consisting of dimensions, shapes, colors, textures, and combinations thereof.

21. A method for mapping out endoluminal gastric reduction, the method comprising:
advancing an endoluminal support into a patient's stomach; and
submucosally marking an interior of the stomach at at least one specified location.

22. The method of claim 21, wherein submucosally marking the interior further comprises approximating the interior and the endoluminal support.

23. The method of claim 21, wherein submucosally marking the interior further comprises submucosally injecting at least one marking element into a wall of the stomach.

24. The method of claim 18, wherein submucosally marking the interior further comprises submucosally marking the interior with at least one marking element chosen from the group consisting of dye, bulking agents, spheres and combinations thereof.

25. A method for mapping out endoluminal gastric reduction, the method comprising:
advancing a radiofrequency endoluminal support into a patient's stomach; and
exposing an interior of the stomach to radiofrequency energy from the support at at least one specified location, wherein said exposure creates a visible marking.

26. The method of claim 25 further comprising approximating the interior of the stomach and the endoluminal support.

27. The method of claim 25, wherein exposing the interior to radiofrequency energy comprises locally burning a mucosa layer of the interior of the stomach.

28. A method for mapping out endoluminal gastric reduction, the method comprising:
advancing an endoluminal support into a patient's stomach; and
marking an interior of the stomach at specified locations with at least one peg delivered from the endoluminal support.

29. The method of claim 28 further comprising approximating the interior of the stomach and the endoluminal support.

30. The method of claim 28 further comprising marking the interior with surgical mesh.

31. A method for mapping out endoluminal gastric reduction, the method comprising:
advancing an endoluminal support into a patient's stomach; and
detecting indicia of the endoluminal support to map out endoluminal gastric reduction.
